(19) United States
(12) Patent Application Publication
(10) Pub. No.: US 2007/0270899 A1
(43) Pub. Date: Nov. 22, 2007

(54) IMPLANT FOR SPREADING THE NASAL WINGS

(76) Inventors: Daniel aWengen, Binningen (CH); Uwe Steinhardt, Hirrlingen (DE)

Correspondence Address: STRIKER, STRIKER & STENBY, 103 EAST NECK ROAD, HUNTINGTON, NY, 11743, US

(21) Appl. No.: 11/748,779

(22) Filed: May 15, 2007

(30) Foreign Application Priority Data
May 17, 2006 (DE) 10 2006 023 058.2

(51) Int. Cl. A61M 29/00; A61F 2/18

(52) U.S. Cl. 606/199; 623/10

(57) ABSTRACT

A roof-shaped implant for spreading the nasal wings, which implant is attachable to the triangular cartilage of the human nose and is made from an initially flat strip that has been bent to a roof shape, has a dorsal section of the implant above the dorsum of the nose is flat or curved so as to be only slightly angled towards the plateau region of the triangular cartilage at an angle of spread of ω>160 °, or curved around the plateau region of the triangular cartilage in a barrel shape at a radius of curvature of r>4 cm, preferably r>10 cm, and two lateral sections extend so as to be parallel in relation to the respective nasal wing at an angle φ of more than 50° towards the flat dorsal section so as to be canted downwards. In this way without incurring major production expenditure it is possible to achieve a particularly good geometric match of the implant to the shape of the human triangular cartilage. In particular, after the operation no hollow spaces arise between the implant and the triangular cartilage.

### CROSS-REFERENCE TO A RELATED APPLICATION

The invention described and claimed hereinbelow is also described in German Patent Application DE 10 2006 023 058.2-55 filed on May 17, 2006. This German Patent Application, whose subject matter is incorporated here by reference, provides the basis for a claim of priority of invention under 35 U.S.C. 119(a)-(d).

### BACKGROUND OF THE INVENTION

The invention relates to a roof-shaped implant for spreading the nasal wings, which implant is attachable to the triangular cartilage of the human nose and is made from an initially flat strip that has been bent into a roof shape.

Such an implant for spreading the nasal wings is, for example, known from EP 1 475 056 A1.

Placing an implant, i.e. a piece of fabric or a material that as a rule is exogenous, i.e. foreign to the body, in the human body is a method that has long been known in medical technology, which method is carried out in many variations to rectify or remedy functional deficiencies of various body parts and/or psychological impairments.

Spreading the nasal wings can, for example, be indicated in the case of stenosis, i.e. narrowing, of the nasal valve, or in the case of a collapse of the soft tissue of the nasal wings.

A simple and known method of spreading the nasal wings involves the use of an adhesive plaster that provides nasal support, which plaster is in particular also used by high-performance athletes in order to improve their nasal breathing. However, this approach is not suitable for permanent application. With regular application of adhesive plaster that provides nasal support, skin problems can arise as a result of the adhesive. Furthermore, a nasal plaster negatively affects the external appearance of a person.

Therefore, for permanent spreading of the nasal wings, surgical methods are known in which cartilage is used to stabilise the lateral nasal soft tissue. However, the results are not always satisfactory either visually or functionally. Moreover, this approach is associated with still further disadvantages. Since preferably the body's own cartilage is used for stabilisation, this step must be preceded with the removal of cartilage, preferably from the patient's ear or nasal septum; a procedure which is associated with expenditure of time and an additional risk to the patient. It has often been shown that the part put in place has insufficient intrinsic tension to keep open the caudal air space of the nose in a satisfactory manner. The method can even be counterproductive because the parts put in place can push the soft tissue inward, thus resulting in further nasal stenosis.

Metal implants are also used for spreading the nasal wings. To this effect, an aperture is surgically made in the region of the edge of the nasal wing, through which aperture the implant is inserted and attached to the triangular cartilage. Metal implants are significantly stronger and more elastic than cartilage, and have sufficient intrinsic tension to keep the nasal air space open in a permanent and satisfactory manner.

From U.S. Pat. No. 6,322,590 B1 a roof-shaped implant is known that is made from a straight flat metal strip. The two ends of this metal strip comprise perforations in the shape of round holes, with said metal strip being bent from the flat shape to a three-dimensional V-shaped roof form, albeit with a “round tip” of the three-dimensional V.

Other implant arrangements in the nose make possible implants according to EP 1 475 056 A1, in which the flat strip is also bent from the flat shape to a three-dimensional V-shaped roof form, except that said strip already in its flat shape has an angular contour, in particular a V-shaped or trapezoid contour.

As is known, the triangular cartilage of the human nose has a relatively flat or at most a very lightly curved plateau region. All known implants are associated with a disadvantage in that they are curved around the dorsum of the nose to such an extent that in the implanted state in the region of this plateau of the triangular cartilage a hollow space is created between the triangular cartilage and the implant, which hollow space can result in unfavourable healing following implantation, and possibly in uncontrolled proliferation of tissue aggregations. Furthermore, the known implants are geometrically designed such that they do not rest optimally against the flanks of the triangular cartilage in the region of the nasal wings.

### SUMMARY OF THE INVENTION

It is thus the object of the invention, without elaborate technical means, to modify an implant of the type mentioned in the introduction to such an extent that these disadvantages are avoided and that following surgery the implant rests as closely as possible against the triangular cartilage.

In a surprisingly simple manner, which is economical to implement, this object is met in that a dorsal section of the implant above the dorsum of the nose is flat or curved so as to be only slightly angled towards the plateau region of the triangular cartilage at an angle of spread of ω>160°, or curved around the plateau region of the triangular cartilage in a barrel-shape at a radius of curvature of r>4 cm, preferably r>10 cm, and in that two lateral sections of the implant on both sides of the nasal wings extend so as to be essentially parallel in relation to the respective nasal wing at an angle φ of more than 50° towards the flat dorsal section so as to be canted downwards.

In this way a particularly good geometric adaptation of the implant to the (normal) shape of the human triangular cartilage is achieved without major production expenditure. In particular, no hollow spaces between the implant and the triangular cartilage arise after surgery, neither in the region of the plateau of the triangular cartilage nor at the flanks of said triangular cartilage. Instead, over its entire surface facing the triangular cartilage the implant fits closely against said triangular cartilage, which also results in particularly good mechanical retention of the implant on the triangular cartilage.

In a preferred exemplary embodiment of the invention the two lateral sections of the flat strip are widened towards their free ends. As a result of this measure the nasal wings can be spread or stabilised over a large area.

Metals and their alloys are extremely well suited to surgical and orthopaedic implants, because apart from very good biocompatibility said metals and their alloys provide good permanent strength and elasticity. Despite their relatively low density, implants made of materials such a titanium or titanium compounds provide excellent mechanical properties with a long service life. Special steel is also well suited to the above-mentioned purposes. While the implant according to the invention can in principle also comprise a suitable plastic material, it is preferably made of metal, in particular of titanium, a titanium alloy or special steel,

There are several options as far as the shape of the implant is concerned. In a simple embodiment the flat strip from which the implant is made can be bent V-shaped in the flat, wherein expediently the point of the V is rounded. The flat strip can also be of a trapezoid shape and/or can comprise complicated structures with instances of branching, which after the implant has been bent open to its final spatial shape can serve to stabilise for example the dorsum of the nose.

In an advantageous embodiment the flat strip or the finished implant can comprise perforations. In this way on the one hand the weight of the implant is reduced, and on the other hand the fraction of exogenous material that is introduced into the human body as a result of the implant is reduced to the greatest extent possible. Furthermore, the perforations promote the growth of tissue into the implant. The perforations are preferably provided both on the lateral sections of the implant and on the dorsal section situated in between, and are, for example, round holes or elongated holes.

Furthermore, the perforations are used for secure fastening to the triangular cartilage by means of a suture.

In addition to the good biocompatibility of the material used, the flat strip or the implant can also comprise a special coating that is tolerated by the body.

In order to achieve a finely matched shape of the flat strip or of the implant, these can expediently be made using laser technology.

Particularly preferred embodiments of the invention are characterised in that the free ends of the two lateral sections of the implant are canted downwards towards the dorsal section by a still greater angle when compared to that of the remaining parts of the lateral sections. In this way a particularly close fit against the triangular cartilage is achieved.

In advantageous improvements of these embodiments the canting angles of the free ends of the two lateral sections are designed such that in their implanted state the lateral sections establish close spatial contact, in particular in a clamping arrangement that is under tension on both sides and that is preferably symmetrical, with the triangular cartilage, which arrangement results in particularly good seating of the implant.

In further advantageous embodiments of the invention the flat strip or the implant is produced by injection moulding according to the micro injection moulding (MIM) method, which is known per se, for example from WO 00/06327 A2. In this way extremely economical production even of very large batches can be achieved with constant dimensional accuracy, while conventional implants as a rule are hand-made not unlike items of jewelry and are therefore not only relatively expensive to produce but also likely to individually vary in dimensional accuracy.

Lastly, particularly preferred are also embodiments of the invention, in which embodiments the flat strip or the implant is made from a material with superelastic characteristics, preferably from Nitinol so that for example by suitable thermal treatment the implant can be given optimal resilient characteristics relative to the triangular cartilage.

Further characteristics and advantages of the invention are shown in the following detailed description of exemplary embodiments of the invention with reference to the figures in the drawing which shows details that are significant in the context of the invention, as well as in the claims, In variants of the invention the individual characteristics can be implemented individually per se, or taken together in any desired combinations.

The diagrammatic drawing shows exemplary embodiments of the invention, which exemplary embodiments are explained in more detail in the following description.

### BRIEF DESCRIPTION OF THE DRAWINGS

FIG. 1 a perspective view of a first exemplary embodiment of an implant according to the invention, comprising a flat dorsal section;

FIG. 2a a diagrammatic front view of the implant shown in FIG. 1 in a direction parallel to the top edge of the dorsum of the nose;

FIG. 2b a diagrammatic front view of a second exemplary embodiment of an implant according to the invention with a dorsal section that is slightly angled towards the plateau region of the triangular cartilage; and

FIG. 3 a diagrammatic view of a flat blank for an implant according to the invention.

### DESCRIPTION OF THE PREFERRED EMBODIMENTS

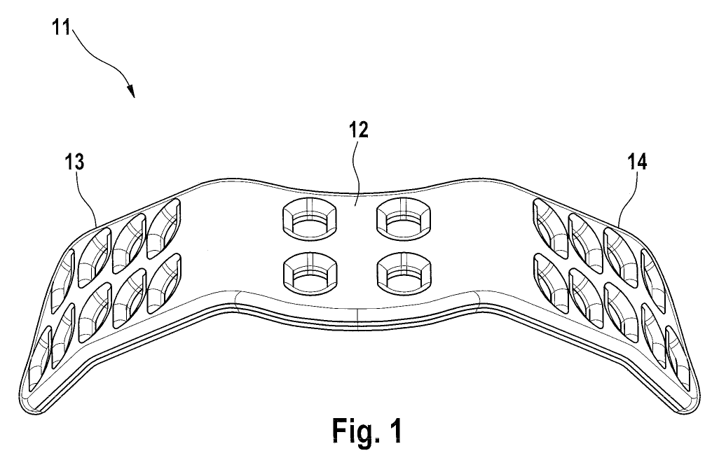

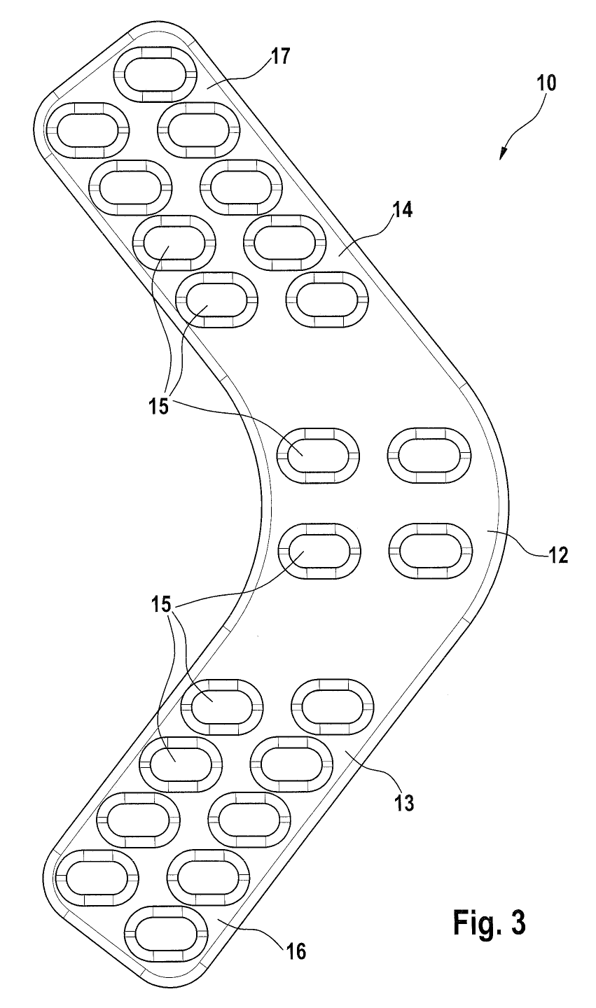

FIG. 1 shows a perspective view of a first possible embodiment of an implant 11 according to the invention. Said implant has been bent from a flat strip 10, as diagrammatically shown in FIG. 3, to form a roof-like spatial shape.

In the flat section already, the strip 10 is bent in a V-like manner and comprises a rounded tip. Said strip 10 comprises a number of regular perforations 15 that help not only to reduce the weight of the implant 11, but also to reduce as far as possible the fraction of exogenous material in the body of a patient. Furthermore, the perforations 15 promote growth of the tissue into the implant 11. The implant 11 is surgically placed underneath the wing cartilage into the nose by means of a so-called open rhinoplasty and is attached to the triangular cartilage by means of a suture. In this process several individual sutures are placed through the perforations and the triangular cartilage and are fixed.

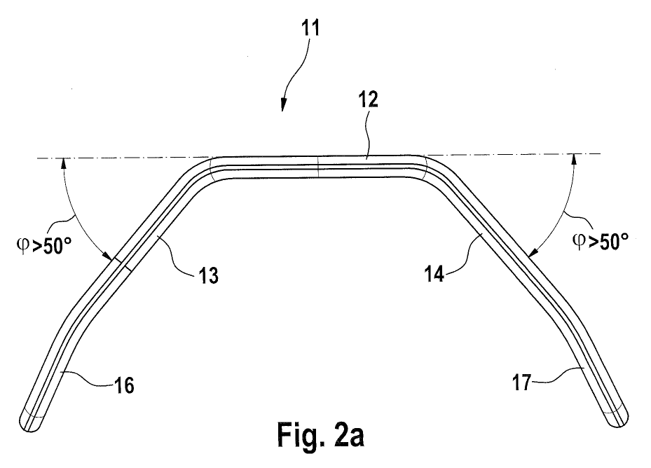

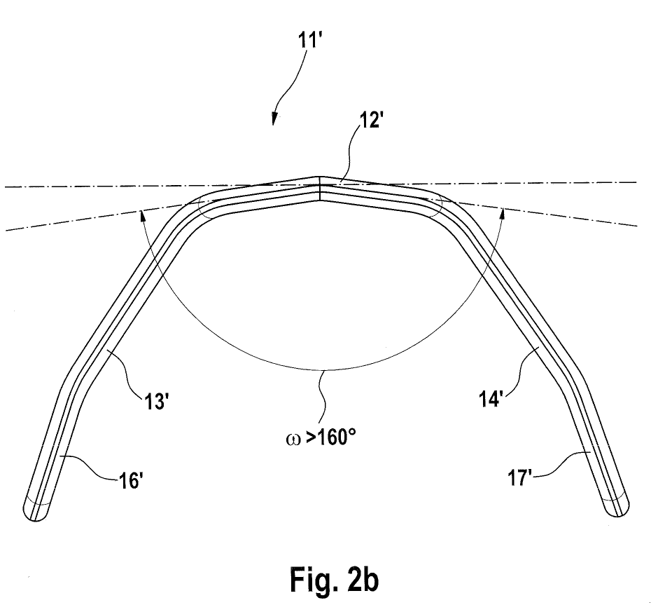

FIGS. 1 and 2a clearly show that the embodiment, shown therein, of the implant 11 according to the invention comprises a flat dorsal section 12, which in the implanted state is arranged above the dorsum of the nose. On both sides of the nasal wings two lateral sections 13, 14 of the implant 11 extend so as to be essentially parallel in relation to the respective nasal wing at an angle φ of more than 50° towards the flat dorsal section 12, canted downwards, as is indicated in particular in FIG. 2a. The free ends 16, 17 of the two lateral sections 13, 14 of the implant 11 extend so as to be canted downwards towards the dorsal section 12 by an angle that is greater still than that of the remaining parts of the lateral sections 13, 14, wherein the canting angles of the free ends 16, 17 are such that the lateral sections 13, 14 in the implanted state are in close spatial contact, in particular in a clamping arrangement, which is under tension on both sides and which is preferably symmetrical, with the triangular cartilage.

However, the dorsal section of the implant according to the invention does not have to be 100% flat. In some embodiments it can also comprise a slight angle or a very slight curvature without the advantages of the invention being altogether lost in this arrangement. On the contrary, in many humans the plateau-shaped region of the triangular cartilage is also not completely flat but instead is slightly curved, so that with such embodiments it is possible to achieve a particularly good geometric match of the implant to the individual features of the patient.

FIG. 2b thus shows an embodiment in which the implant 11′ comprises a dorsal section 12′ that is only very slightly angled towards the plateau region of the triangular cartilage at an angle of spread ω>160°. In this arrangement too the two lateral sections 13′, 14′ of the implant 11′ extend so as to be essentially parallel in relation to the respective nasal wing at an angle φ of more than 50° towards the flat dorsal section 12′ so as to be canted downwards, again ending in free ends 16′, 17′ that are canted downwards towards the dorsal section 12′ at a still greater angle when compared to that of the remaining parts of the lateral sections 13′ 14′.

In further embodiments, not shown in the drawing, instead of the slight angle shown in FIG. 2b the dorsal section of the implant according to the invention can also comprise a very slight barrel-shaped curvature with a relatively large radius of curvature r>4 cm, preferably even r>10 cm around the plateau region of the triangular cartilage.

It will be understood that each of the elements described above, or two or more together, may also find a useful application in other types of constructions differing from the type described above.

While the invention has been illustrated and described as embodied in an implant for spreading the nasal wings, it is not intended to be limited to the details shown, since various modifications and structural changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims:

1. A roof-shaped implant for spreading nasal wings, attachable to a triangular cartilage of a human nose and composed of an initially flat strip that has been bent to a roof shape, the implant comprising a dorsal section to be located above a dorsum of a nose and having a shape selected from the group consisting of a flat shape and a curved shape so as to be only slightly angled toward a plateau region of the triangular cartilage at an angle of spread of ω>160°, or curved around a plateau region of the triangular cartilage in a barrel shape at a radius of curvature of r>4 cm; and two lateral sections to be arranged on both sides of the nasal wings and extending so as to be essentially parallel in relation to a respective nasal wing at an angle φ of more than 50° toward said flat dorsal section so as to be canted downwards.

2. A roof-shaped implant as defined in claim 1, wherein said two lateral sections are widened toward their free ends.

3. A roof-shaped implant as defined in claim 1, wherein the flat implant is composed of metal.

4. A roof-shaped implant as defined in claim 1, wherein the implant is composed of metal selected from the group consisting of titanium, a titanium alloy, and a special steel.

5. A roof-shaped implant as defined in claim 1, wherein the implant has a contour selected from the group consisting of an angled contour and a trapezoid contour with or without instances of branching.

6. A roof-shaped implant as defined in claim 5, wherein the implant has the angled contour which is V-shaped.

7. A roof-shaped implant as defined in claim 1, wherein the implant comprises perforations.

8. A roof-shaped implant as defined in claim 7, wherein the perforations are provided both on said lateral sections and on said dorsal section situated in between.

9. A roof-shaped implant as defined in claim 6, wherein said perforations are formed as holes selected from the group consisting of round holes and elongated holes.

10. A roof-shaped implant as defined in claim 1, wherein the implant comprises a coating that is tolerated by a body.

11. A roof-shaped implant as defined in claim 1, wherein the implant is formed as a laser-produced implant.

12. A roof-shaped implant as defined in claim 1, wherein said two lateral sections have free ends which are canted downwards towards said dorsal section by a still greater angle when compared to an angle of remaining parts of said lateral sections.

13. A roof-shaped implant as defined in claim 11, wherein the canting angles of said free ends of said two lateral sections are configured such that in their implanted state said lateral sections establish close spatial contact in particular in the clamping arrangement that is under section on both sides with a triangular cartilage.

14. A roof-shaped implant as defined in claim 12, wherein said lateral sections are configured such that in their implanted state they establish close spatial contact in the clamping arrangement that is under tension on both sides and is symmetrical, with the triangular cartilage.

15. A roof-shaped implant as defined in claim 1, wherein the implant is formed as a micro injection-molded implant.

16. A roof-shaped implant as defined in claim 1, wherein the implant is composed of a material having a memory effect.

17. A roof-shaped implant as defined in claim 16, wherein the implant is composed of Nitinol.
